(19) United States
(12) Patent Application Publication
(10) Pub. No.: US 2020/0261669 A1
(43) Pub. Date: Aug. 20, 2020

(54) Delivery System for Metered Dose Inhalers

(71) Applicant: ABITHAS, LLC, Chicago, IL (US)

(72) Inventor: Edward E. Lisberg, Chicago, IL (US)

(73) Assignee: ABITHAS, LLC, Chicago, IL (US)

(21) Appl. No.: 16/799,631

(22) Filed: Feb. 24, 2020

Related U.S. Application Data
(63) Continuation-in-part of application No. 16/528,957, filed on Aug. 1, 2019.
Application No. 16/528,957 is a continuation-in-part of application No. 15/057,907, filed on Mar. 1, 2016, now abandoned.
(60) Provisional application No. 62/126,973, filed on Mar. 2, 2015.

(51) Int. Cl. A61M 15/00

(52) U.S. Cl. CPC A61M 15/0065; A61M 15/0088; A61M 2205/583; A61M 15/009; A61M 2205/581; A61M 15/0025

(57) ABSTRACT

An accessory device for delivering medications from attached press-and-breathe metered-dose inhalers (MDIs), including those medications containing hydrofluoroalkane propellants. The device includes a collapsible flexible bag to which is attached a bidirectional mouthpiece and an adaptor that receives the MDI. The mouthpiece contains a reed that functions as an audible signal. The adaptor positions the MDI at an angle to direct the aerosol spray toward the center of the bag. When triggered, the MDI discharges the aerosolized medication into the center of the bag, which medication may then be inhaled by the user through the mouthpiece. The user's inhalation causes the bag to collapse. The reed emits an audible sound if the user inhales above a pre-determined flow rate. A cap seals a gap between the adaptor and an attached canister, making the device in combination with the MDI into a closed-bag system.

### CROSS REFERENCE TO RELATED APPLICATIONS

This application is a continuation-in-part (CIP) of U.S. application Ser. No. 16/528,957, filed Aug. 1, 2019, which is a CIP of U.S. application Ser. No. 15/057,907 filed Mar. 1, 2016, which is based on and claims the priority of U.S. provisional patent application Ser. No. 62/126,973 filed Mar. 2, 2015 (“the Provisional Application”), the entire contents of each of which are hereby fully incorporated herein by reference for all purposes.

### FIELD OF THE INVENTION

This invention relates to systems for delivering aerosolized medication from metered-dose inhalers.

### BACKGROUND

Delivery of pharmaceuticals via inhalation has long been considered the standard of care for the treatment patients with acute and chronic respiratory diseases such as asthma and chronic obstructive pulmonary disease (“COPD”). Press-and-breathe metered-dose inhalers (“MDIs”) have become the mainstay of inhaled treatment for such patients and are widely known and used by the medical profession. Experience clearly shows that while widely prescribed, many patients cannot or will not use MDIs as intended.

Suboptimal MDI inhalation techniques contribute to poor lung deposition of medication, poor disease control, adverse asthma and COPD outcomes, and increased medical costs. Studies demonstrate the inability of both patients and healthcare providers to use MDIs properly or effectively.

A number of devices (so-called “spacers”) have been proposed to assist in MDI use. In laboratory test conditions, many Spacers have appeared to improve MDI aerosol delivery to the lower airways; however, outside of the laboratory, experience demonstrates that many patients cannot consistently use these spacers as intended. A major factor contributing to the lack of MDI spacer user consistency is that device users cannot determine if they have consistently both (1) fully inhaled the complete dose following MDI actuation, and (2) inhaled at a sufficiently low inspiratory flow rate necessary for effective delivery of aerosolized medication from MDIs.

Once a MDI canister is triggered, the most important patient-centered factors that relate to optimal lung delivery of medication are: (1) initiation of inhalation prior to 80% of total lung capacity (within approximately the first 1-2 seconds after medication is aerosolized), and (2) that the user generates a sufficiently low inspiratory flow rate to effectively deliver proper sized optimal aerosol particles into the lung alveoli. The subjective terms “long” or “slowly” are common instructions by manufacturers on MDI medication inserts, but these terms have been of little value in ensuring proper patient inhalation technique. Spacer and MDI devices which lack an effective inspiratory flow signal or fail to provide effective visual and auditory feedback regarding complete dose inhalation may result in medication dosing that is not constant dose-to-dose or patient-to-patient.

To aid in this discussion, a conventional pressurized press-and-breathe MDI is described with reference to FIG. 1. As illustrated in FIG. 1, an MDI 100 includes an original manufacture assembled pressurized canister 102 containing a drug/propellent and possibly excipients mixture 104. The canister 102 is mounted in an actuator 106. A metering valve stem 108 extends out from the canister 102 and is received in an actuator seat 110. The metering valve dispenses the drug substance/propellent 104 contained within the canister 102, through an actuator nozzle 112, generating a mist or plume 114 which exits through the actuator exit tube 116.

In order to deliver a correct, accurate, and consistent drug doses, manufacturers carefully match canisters, metering valve stems (part of the canister) with actuators. For example, after chlorofluorocarbons (“CFCs”) were banned, the change in propellants (to hydrofluoroalkanes (“HFAs”)) required design of new pressurized press-and-breathe MDI medication canisters, metering valve stems, and actuators.

As should be appreciated, an MDI medication canister is distinct from the actuator within which it is mounted. However, separating MDI medication canisters from the actuators with which they were specifically matched will lead to inconsistent and arbitrary doses. Systems that separate a medication canister from its actuator are not capable of ensuring dose accuracy per puff or puff-to-puff dose equivalency. This is one reason the U.S. Food and Drug Administration (FDA) considers each pressurized MDI drug canister and associated actuator device as a unique “combination product,” where each such product is subject to individual approval and regulation.

There is a gap (denoted G) between the inside walls of the actuator 106 and the canister 102. During operation of the MDI, air enters the gap (G) and may mix with the dispensed drug substance/propellant 104. Different MDI drug canister and actuator combinations may have different gap sizes, and may thus have different amounts of external air that may mix with the dispensed drug substance/propellant 104.

It is therefore desirable and an object hereof to provide a delivery system for metered dose inhalers that works with an MDI device without separating the drug canister and its associated actuator device.

It is desirable and an object hereof to provide a device to be used with intact, originally manufactured press-and-breathe MDI combination products to assist in the proper delivery of aerosolized medication from those products.

### SUMMARY

The present invention is specified in the claims as well as in the below description. Preferred embodiments are particularly specified in the dependent claims and the description of various embodiments.

One general aspect includes an accessory delivery device for press-and-breathe metered dose inhalers. The device may also include a flexible collapsible bag having an open top end and a closed bottom end. The device may also include a top end cap having a top surface, and having a first opening and a second opening in the top surface, the top end cap being connected to the open top end of the flexible collapsible bag. The device may also include a tubular mouthpiece having a proximal end suitable to place in a user's mouth, and a distal end mounted in the first opening of said top surface, the tubular mouthpiece in fluid communication with the flexible collapsible bag. The device may also include a warning indicator constructed and adapted to provide an audible signal based on a predetermined inhalation flow rate through the tubular mouthpiece is exceeded. The device may also include an upstanding collar in the second opening in the top end cap, the upstanding collar being disposed at a fixed angle offset from perpendicular to the top surface of the top end cap. The device may also include an adaptor mounted on the upstanding collar, the adaptor having a centrally disposed flexible member, an opening in the centrally disposed flexible member adapted to receive a first portion of an actuator of a press-and-breathe metered dose inhaler. The device may also include a cap constructed and adapted to seal a gap between an inner wall of the actuator and a canister inserted in the actuator. The device may also include where, when activated, the press-and-breathe metered dose inhaler dispenses an aerosol spray containing a drug through the adaptor in a direction away from the top end cap and into the flexible collapsible bag.

Implementations may include one or more of the following features, alone and/or in combination(s):
The device wherein, when attached to the MDI, the cap prevents air flow through the gap between the inner wall of the actuator and the canister.
The device wherein, when attached to the MDI, the cap provides access to a top portion of the canister to allow user activation of the MDI.
The device, further comprising a tether attached to the cap at a first end of the tether, wherein the tether comprises a ring at a seconds end thereof, said ring connecting said tether to said collar.
The device may include a flexible sealing member mounted in the centrally disposed flexible member having a slot to receive the press-and-breathe metered dose inhaler in fluid tight engagement.
The device may include a bottom end cap sealing the bottom end of the flexible collapsible bag.
The device where the top end cap and bottom end cap may include high density polyethylene.
The device where the flexible collapsible bag is low density polyethylene.
The device where the flexible collapsible bag is metallocene low density polyethylene.
The device where the warning indicator produces an audible sound when a flow rate of air passing through the tubular mouthpiece exceeds the predetermined inhalation flow rate.
The device where the warning indicator produces a whistling sound when the flow rate of air passing through the tubular mouthpiece from the flexible collapsible bag exceeds the predetermined inhalation flow rate.
The device where the warning indicator may include a reed that vibrates to produce the audible sound when a flow rate of air passing through the tubular mouthpiece exceeds the predetermined inhalation flow rate.
The device where the mouthpiece has locking tabs in the distal end and the first opening has slots to receive the locking tabs where the mouthpiece is removably attached to the first opening.
The device where the collar has keyways and the adaptor has internal keys that are received in the keyways to align the adaptor with the top end cap having the opening in the centrally disposed flexible member aligned to receive the press-and-breathe metered dose inhaler.
The device where the flexible collapsible bag gives a visual indication that an appropriate amount of the aerosol spray containing the drug has been inhaled when the bag is collapsed.
The device where the device may include a channel in the press-and-breathe metered dose inhaler adaptor that receives the collar to securely attach the press-and-breathe metered dose inhaler adaptor to the collar.
The device where the flexible collapsible bag has a volume of between 690-700 cubic cm. when in a fully expanded position.
The device where the fixed angle of the collar to the top surface of the top end cap is between 8 to 18 degrees.
The device where an airtight seal is provided between the top end cap and the open top end of the flexible collapsible bag.
The device where the airtight seal is provided by a seal surrounding the top end cap.
The device where the bottom end of the flexible collapsible bag is sealed to prevent passage of air into or out from the flexible collapsible bag from the bottom end.
The device where the warning indicator is mounted in the tubular mouthpiece.
The device where the device may include: a filter within the tubular mouthpiece.
The device where the Tubular mouthpiece protrudes vertically from the top surface of the top end cap.
The device where the adaptor is constructed and adapted to fit multiple different sized inhaler actuators.
The device where the fixed angle is offset from perpendicular to the top surface of the top end cap when the device is in use.
The device where the first portion of the actuator may include an actuator exit tube of the actuator.

Thus, in some aspects, I describe a device to be used with intact, originally manufactured press-and-breathe MDI combination products to assist in the proper delivery of aerosolized medication from MDIs.

Embodiments hereof provide a simple, efficient, easy to use device for patients to consistently deliver containing medications from MDIs. Embodiments ensure consistent puff-to-puff delivery of inhaled medications via an adaptor optimized for MDI medications, and an effective inspiratory flow signal.

The above features along with additional details of the invention are described further in the examples herein, which are intended to further illustrate the invention but are not intended to limit its scope in any way.

### DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 illustrates aspects of a conventional press-and-breathe MDI showing a drug/propellant-containing canister seated in an actuator;

FIG. 2 depicts aspects of an exploded view of a delivery device according to exemplary embodiments hereof;

FIGS. 3 and 4 are top and top perspective views, respectively, of a top end cap of a device according to exemplary embodiments hereof, with a mouthpiece and adaptor attached;

FIG. 5A is a side view of the mouthpiece shown in FIGS. 2-4;

FIG. 5B is a rear perspective view of the mouthpiece of FIG. 5A showing aspects of a screen component of the mouthpiece;

FIGS. 6A and 6B are side views of an alternate mouthpiece according to exemplary embodiments hereof;

FIGS. 7 and 8 are top and bottom views, respectively, of a reed attachment according to exemplary embodiments hereof;

FIGS. 9 and 10 are top and bottom perspective views, respectively, of an adaptor according to exemplary embodiments hereof;

FIG. 11 is a cross sectional view of exemplary the adaptor, taken along lines X-X of FIG. 9;

FIG. 12 is a top perspective view of a top end cap according to exemplary embodiments hereof;

FIG. 13 is cross-sectional view of the exemplary cap of FIG. 12;

FIGS. 14 and 15 are bottom and top views, respectively, of a bottom end cap according to exemplary embodiments hereof;

FIGS. 16 and 17 are front perspective views of a collapsible flexible bag according to exemplary embodiments hereof;

FIG. 18 is a front perspective view of an assembled device according to exemplary embodiments hereof; and

FIG. 19 is a perspective view of a device according to exemplary embodiments hereof, with a press-and-breathe MDI actuator inserted into the adaptor;

FIG. 20 is a bottom view of the top end cap with an MDI actuator inserted;

FIG. 21 is a front perspective view of an assembled device according to exemplary embodiments hereof, using the mouthpiece of FIGS. 6A-6B;

FIGS. 22A-22C depict aspects of a cap according to exemplary embodiments hereof;

FIGS. 23A-23C depict aspects of a tether for a cap according to exemplary embodiments hereof;

FIGS. 24A and 24B depict aspects of an exemplary delivery device using a tethered cap according to exemplary embodiments hereof; and

FIGS. 25A-25B shows the exemplary device of FIGS. 24A and 24B in use.

### DESCRIPTION OF THE PRESENTLY PREFERRED EXEMPLARY EMBODIMENTS

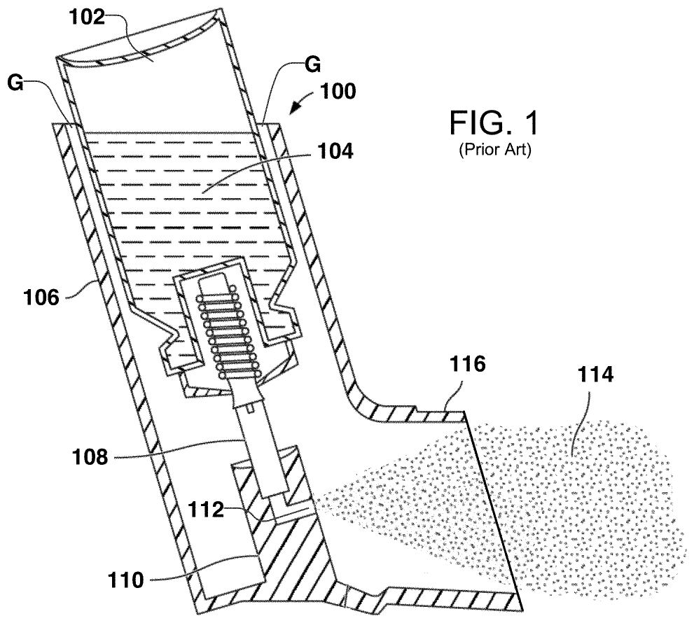

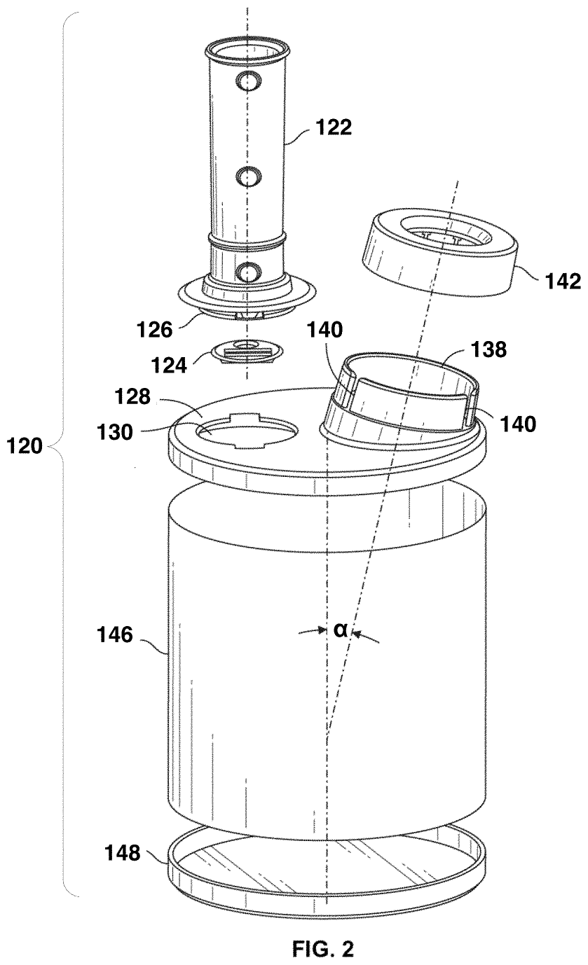

FIG. 2 depicts aspects of an exploded view of an exemplary delivery device 120 for press-and-breathe metered dose inhalers (e.g., the MDI 100 of FIG. 1).

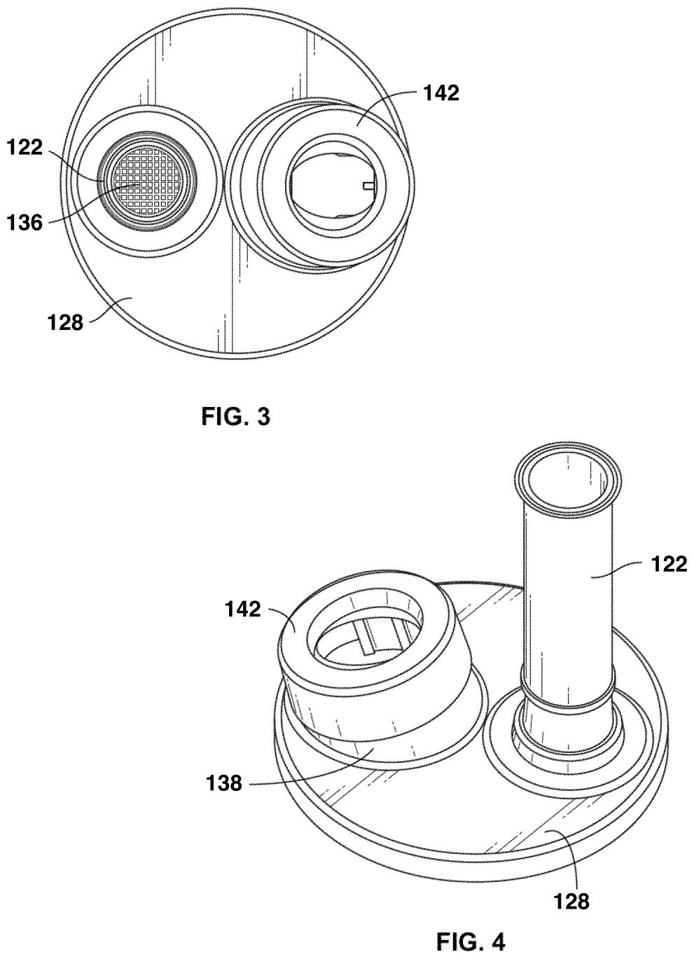

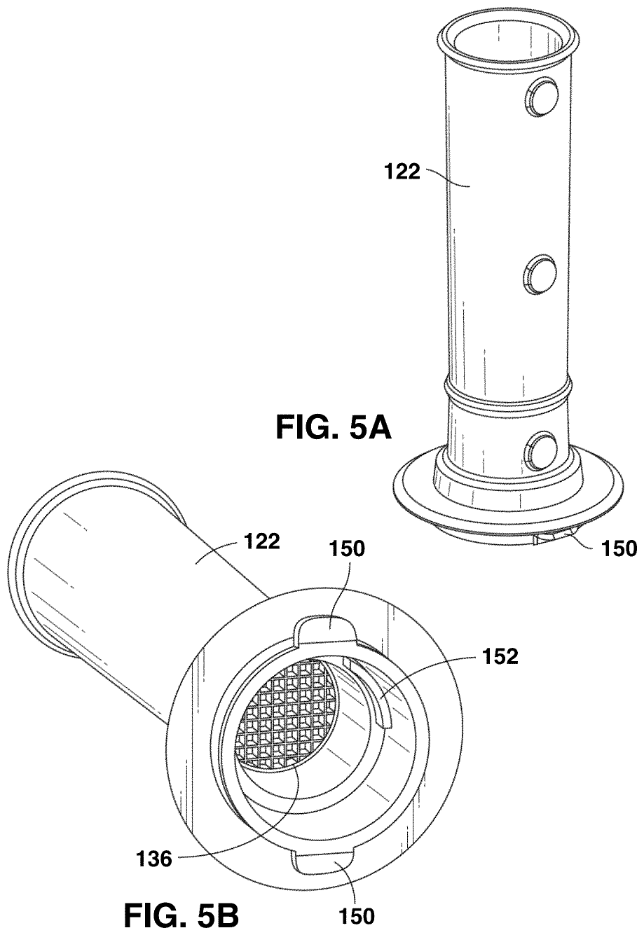

A mouthpiece 122 is inserted into a top end cap 128 through an opening 130. The opening 130 may have two opposite rectangular slots 132 to receive corresponding locking tabs 134 at the lower end 126 of the mouthpiece 122 (seen in FIG. 5B).

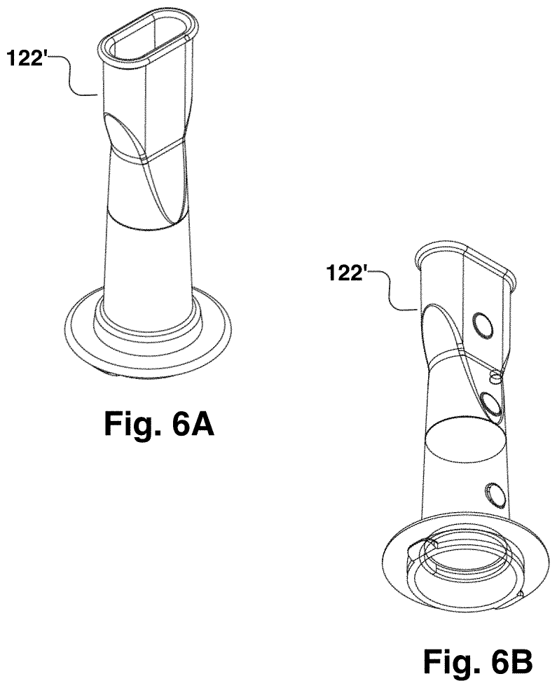

The mouthpiece 122 may contain a filter in its central channel (e.g., a screen 136, best seen in FIGS. 3 and 6).

The top end cap 128 has an upstanding collar 138 angularly disposed with respect to the top planar surface of the top end cap 128. The collar 138 may be formed separately from the top end cap 128 and then attached thereto, or the collar 138 and cap 128 may be integrally formed.

The mouthpiece 122 may have a reed attachment 124, described in greater detail below, inserted into a lower end 126 thereof.

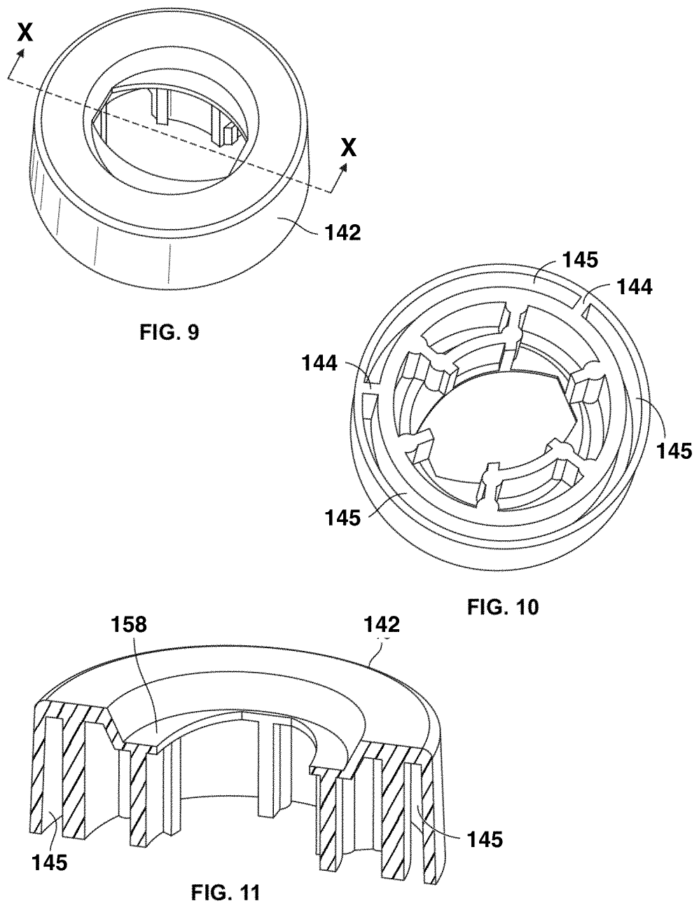

A pair of vertically disposed keyways 140 are cut into the wall of the collar 138. An adaptor 142 (seen also FIGS. 9-11) is mounted on the collar 138. Keys 144 (best seen in FIG. 10) may be received in the keyways 140 to properly align the adaptor 142 with the collar 138 so that the MDI's actuator (e.g., actuator 106 in FIG. 1) is properly positioned for use in the adaptor 142. A channel 145 in the MDI adaptor (best seen in FIG. 11) receives the collar 138 in tight engagement to firmly, but releasably retain the adaptor 142 on the collar 138.

The top end cap 128 is preferably made from high density polyethylene (HDPE) and is substantially flat except for the perimeter that may have an upstanding or tapered edge or other surface imperfections due to the plastic molding process.

A collapsible flexible bag 146 is located below the top end cap 128.

At the bottom of the bag 146 is a bottom end cap 148. The top end cap 128 has a circumferential collar that closely receives the top of the bag 146, and the bottom end cap 148 has a similar circumferential collar that receives the bottom of the bag 146.

The bag 146 is preferably made from a low density metallocene polyethylene (“LDPE”). However other similar materials exhibiting the same characteristics may also be used.

The low density metallocene polyethylene exhibits at least two important characteristics. First it allows the bag 146 to attach to the top end cap 128 and bottom end cap 148 which are made from high density polyethylene (“HDPE”). This creates an airtight seal between the top and bottom end caps 128 and 148 and the bag 146. Second the metallocene LDPE exhibits antibacterial properties.

In a presently preferred implementation, the volume of the bag 146, when in the expanded position, should preferably be approximately 690-700 cubic cm. (i.e., between 42.11 and 42.72 cubic inches).

FIGS. 3 and 4 illustrate aspects of an exemplary mouthpiece 122 and adaptor 142, according to exemplary embodiments hereof, mounted on the top end cap 128. Airtight seals are preferably provided between the mouthpiece 122 and the top end cap 128 and between the adaptor 142 and the collar 138.

With reference now to FIGS. 5A and 5B, locking tabs 150 are positioned at opposite sides of the bottom of the mouthpiece 122. As shown in FIG. 5B, the mouthpiece 122 may include an internal collar 152 for a reed attachment, to receive and correctly position the reed attachment 124 within the bottom of the mouthpiece 122.

FIGS. 6A and 6B are side views of an alternate mouthpiece 122′ according to exemplary embodiments hereof. The mouthpiece 122′ may connect to the top end cap 128 in the same way as mouthpiece 122, and may have similar internal structure to mouthpiece 122, except that the top end of mouthpiece 122′ tapers to an oval shape for easier insertion into a user's mouth.

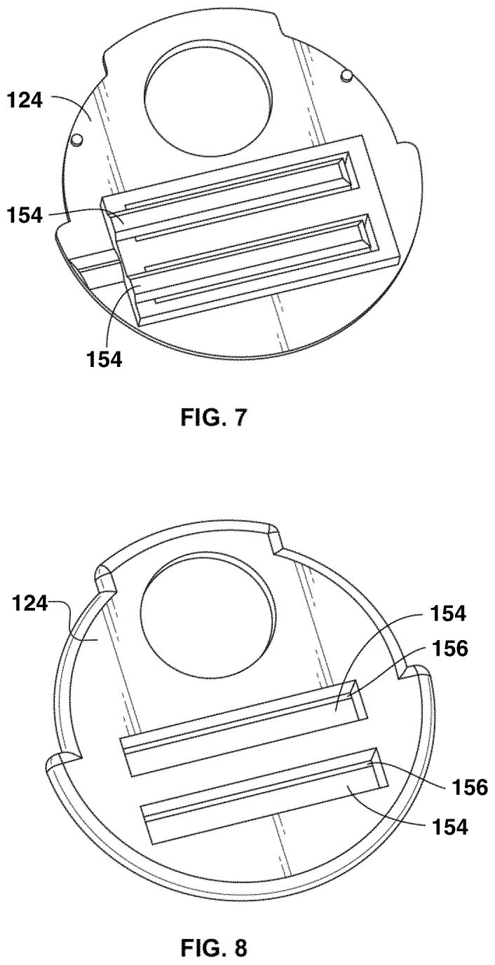

As shown, e.g., in FIGS. 7 and 8, a reed attachment 124 according to exemplary embodiments hereof may include a pair of vibrating members 154 mounted in slots 156. One end of the each of the vibrating members 154 is fixed to the reed body while the opposite end is free to vibrate. The purpose of the reed attachment is to produce an audible sound if the inhalation air flow through the mouthpiece exceeds a preset level. Those of skill in the art will understand, upon reading this description, that other mechanisms and/or types of reed designs may be used.

FIGS. 9-11 illustrate aspects of an adaptor 142 according to exemplary embodiments hereof. The adaptor 142 is preferably made from a flexible material that is sufficiently rigid to retain its shape when inserted onto the collar 138. The adaptor 142 has an innermost ring 158 (seen also in FIG. 13) with a centrally disposed slot that is flexible enough to receive various size MDI actuator exit tubes 116. The MDI actuator is snugly received in the slot in the innermost ring 158 so that there are no gaps between the innermost ring and the MDI actuator thus forming a substantially airtight seal between the innermost ring 158 and the adaptor 142.

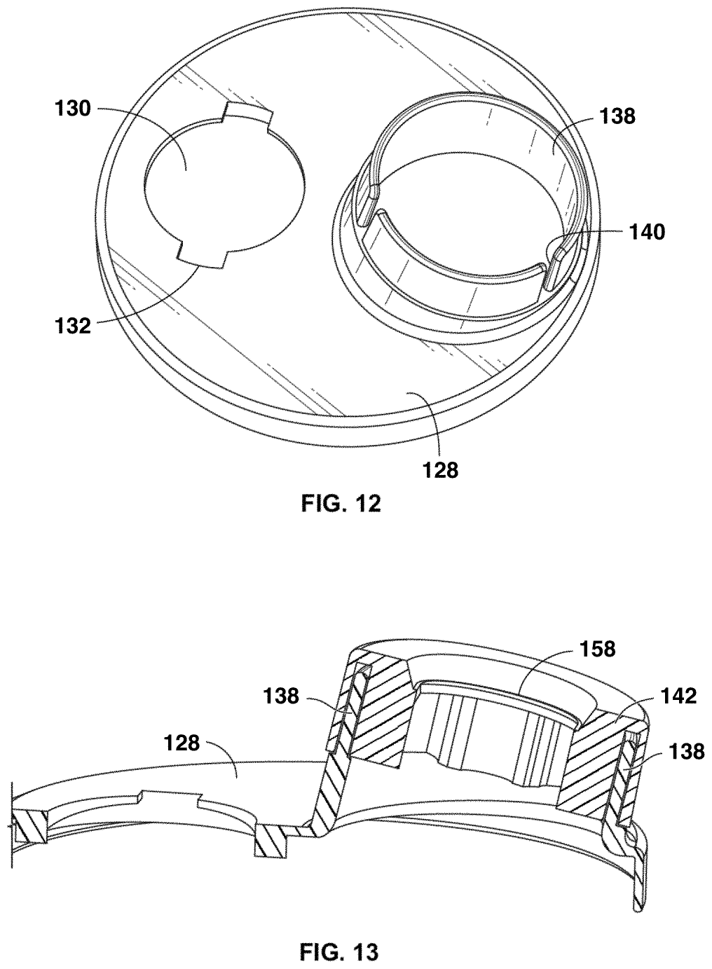

As shown, e.g., in FIGS. 2 and 12, an opening 130 with slots 132 is cut in the top end cap 128. These slots 132 receive the corresponding locking tabs 150 in the bottom of the mouthpiece 122 (and best seen in FIG. 6). Once the locking tabs 150 are inserted through the slots 132, the mouthpiece may be rotated so that the locking tabs 150 firmly lock the mouthpiece to the top end cap 128.

As also shown, e.g., in FIGS. 2 and 12, the collar 138 has keyways 140 constructed to receive the corresponding keys 144 (seen best in FIG. 10) in the underside of the adaptor 142.

FIG. 13 is a cross-sectional view of a portion of an exemplary adaptor 142 mounted to a collar 138 which is mounted to or integrally formed with the top end cap 128.

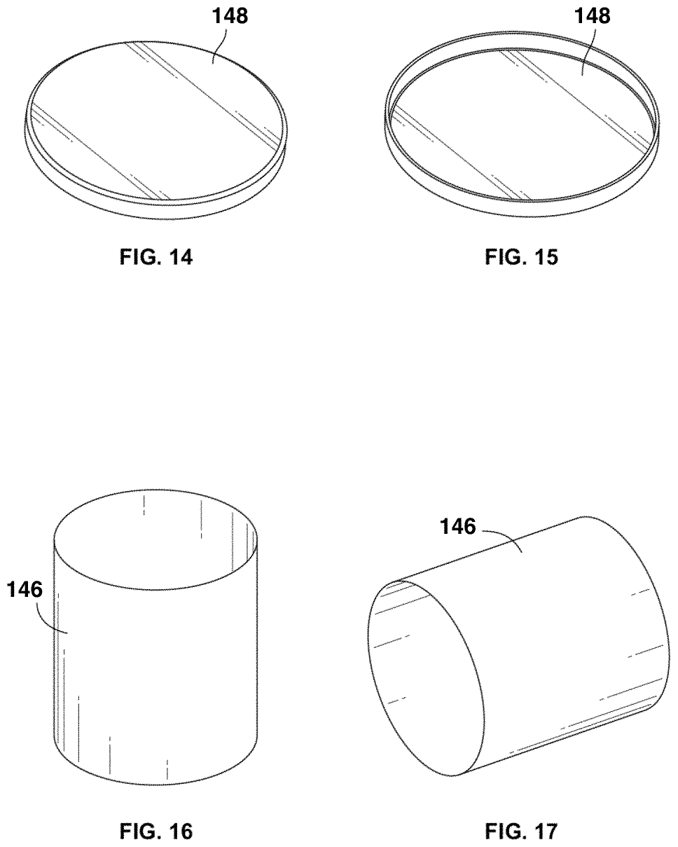

FIGS. 14 and 15 are bottom and top views, respectively, of a bottom end cap 148 according to exemplary embodiments hereof. As seen, e.g., in FIG. 15, the bottom end cap 148 preferably has is an upstanding collar or rim that closely receives, in an airtight fitting, the bottom of the bag 146.

FIGS. 16 and 17 illustrate aspects of the cylindrical shape of the bag 146 according to exemplary embodiments hereof, with the bag 146 being shown in the drawings in its fully expand form.

FIG. 18 illustrates an assembled delivery device 200 according to exemplary embodiments hereof. As shown in the drawing in FIG. 18, the mouthpiece 122 and adaptor 142 are fitted onto the top end cap 128. Inside of the mouthpiece 122, and not visible from the outside, are the reed 124 and screen 136. The bag 146 is securely fitted to the top end cap 128 and bottom end cap 148.

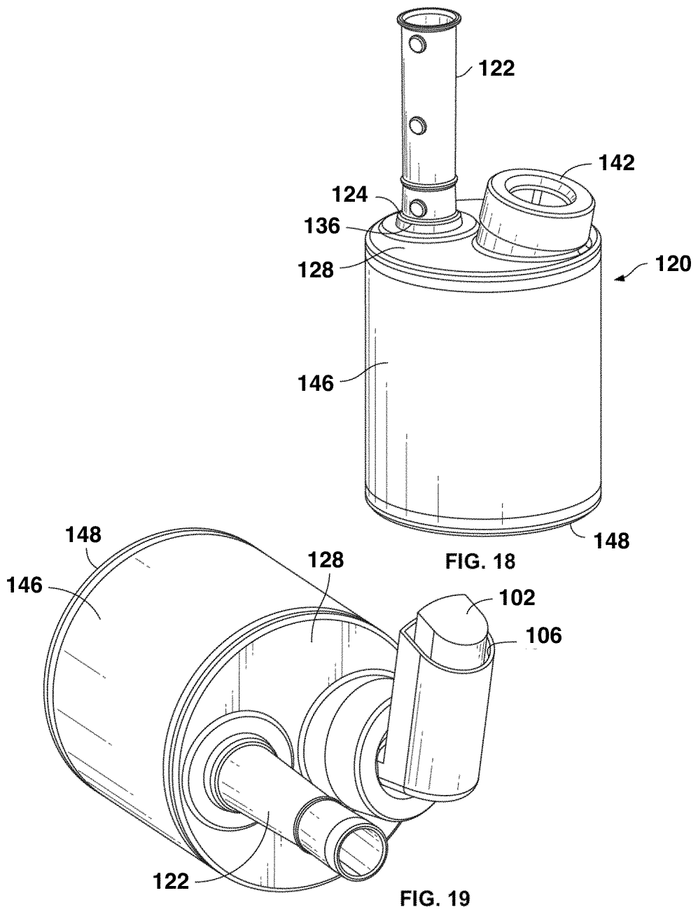

FIG. 19 shows an exemplary device 200, similar to FIG. 18, with an MDI (e.g., the MDI 100 of FIG. 1) inserted into the adaptor 142.

As seen in the drawings, e.g., in FIGS. 2, 4, 13, 18, and 19, the collar 138 is angularly disposed at an angle (denoted “a”) with respect to the center line along the axis of the bag 146. Since the adaptor is inserted into the collar, the adaptor will also be at an offset angle (“a”) from perpendicular to the top end cap 128. Those of skill in the art will understand, upon reading this description, that configuring the adaptor 142 at an offset angle from perpendicular to the top end cap 128 may improve ease of use of the device and may optimize aerosol delivery into the collapsible bag (See, e.g., the Provisional Application, item 11, pg. 5).

In some presently preferred implementations, the angle α is between 8° to 18°, preferably 13°.

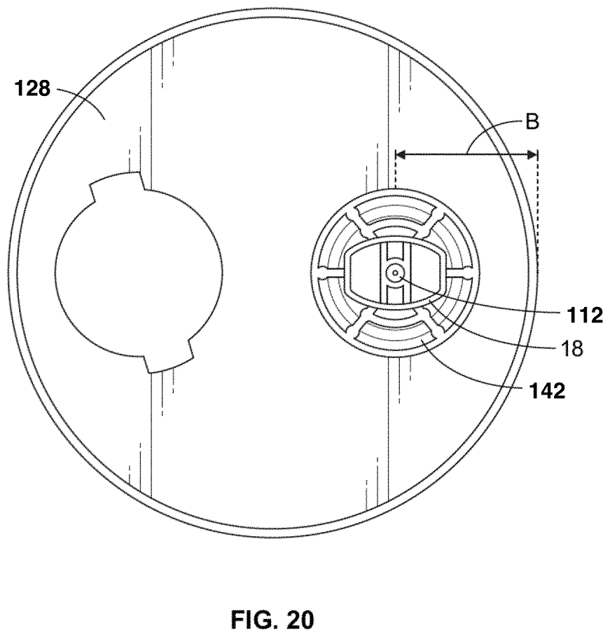

The angle α may be determined by the location of the collar 138 from the perimeter of the end cap 128 so that the spray or plume of medication from the canister is disbursed into the center of the bag 146 when the bag 146 is in the expanded position in a direction toward the center of the bottom end cap 148. This optimizes spray plume distribution within the bag 146 and minimizes the spray hitting the side walls of the bag 146. For example, when the device 200 is held horizontally, and with a diameter of the end cap being 3.085 inches, the distance from the circumference of the end cap to a line extending horizontally from the center of actuator nozzle 112 (see FIG. 1) is between 0.984 inches to 1.122 inches as denoted in FIG. 20 by the letter “B”.

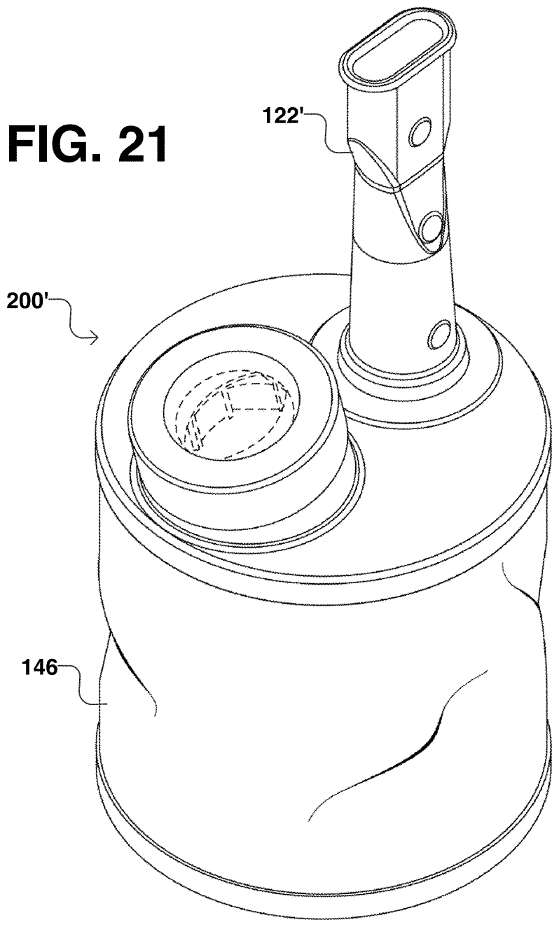

FIG. 21 is a front perspective view of an assembled device 200′ according to exemplary embodiments hereof, using the mouthpiece 122′ of FIGS. 6A-6B. The device 200′ is shown in the drawing with the flexible bag 146 being partially collapsed.

A Cap

In order to control the amount of medication dispensed, it is preferable and desirable for the device 120 in combination with a particular MDI to form a closed-bag system. However, as noted above, with reference to FIG. 1, an MDI has a gap (G in FIG. 1) between the canister 102 and the inner wall of the actuator 106. The gap allows air to enter into the actuator body and means that the combination device/MDI does not form a closed-bag system. Furthermore, as also noted above, manufacturers vary the size of the gap from product to product.

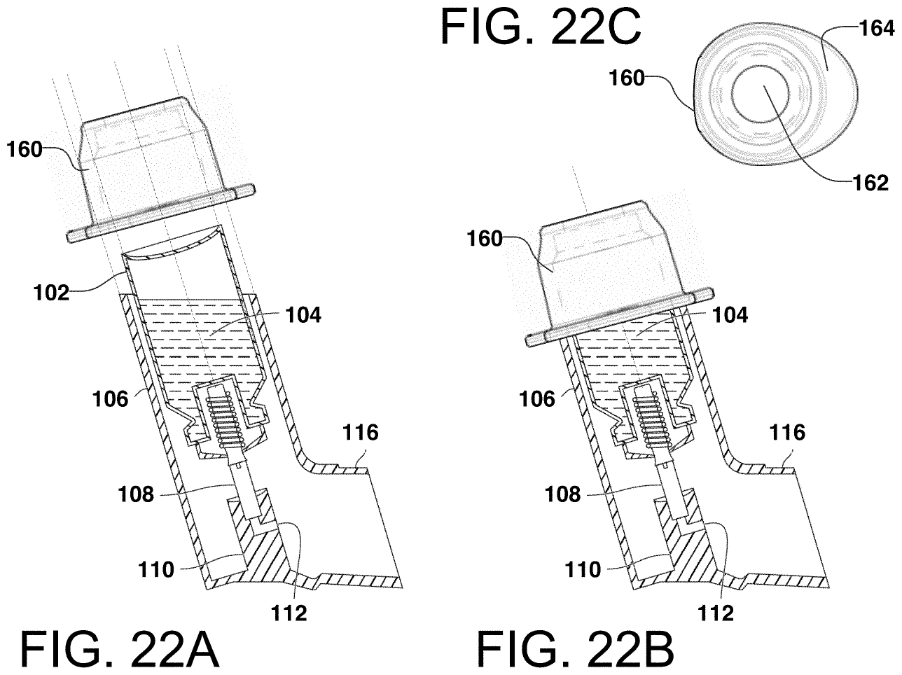

Accordingly, with reference now to FIGS. 22A-22C, embodiments hereof may incorporate a cap 160 sized to fit over the top of an actuator 106 with an installed canister 102. The cap 160 covers the top of the combined actuator/canister, closing or sealing the gap (G) between the canister 102 and the inner wall of the actuator 106. Preferably the cap 160 forms an air-tight seal of the gap (G). The combination of a device 120 with an actuator/canister having cap 160 thereon forms a closed-bag system.

FIG. 22A shows a cap 160 and an actuator/canister combination, and FIG. 22B shows the cap 160 installed or positioned on the actuator/canister combination.

As shown best in FIG. 22C, the cap 160 may have a hole 162 in the top thereof to aid in a user triggering the inhaler. However, the hole 162 preferably does not allow air to enter the system once the cap is installed or positioned on the actuator/canister combination. The cap is shown in the drawings as being circular in shape. However, those of skill in the art will understand, upon reading this description, that the cap may be any shape, including rectangular or some other polygonal shape, as long as it fits on an actuator/canister combination and closes or seals the gap between the canister and the inner wall of the actuator.

The cap 160 may have an outer ridge portion 164 to aid in removal of the cap from the actuator/canister combination.

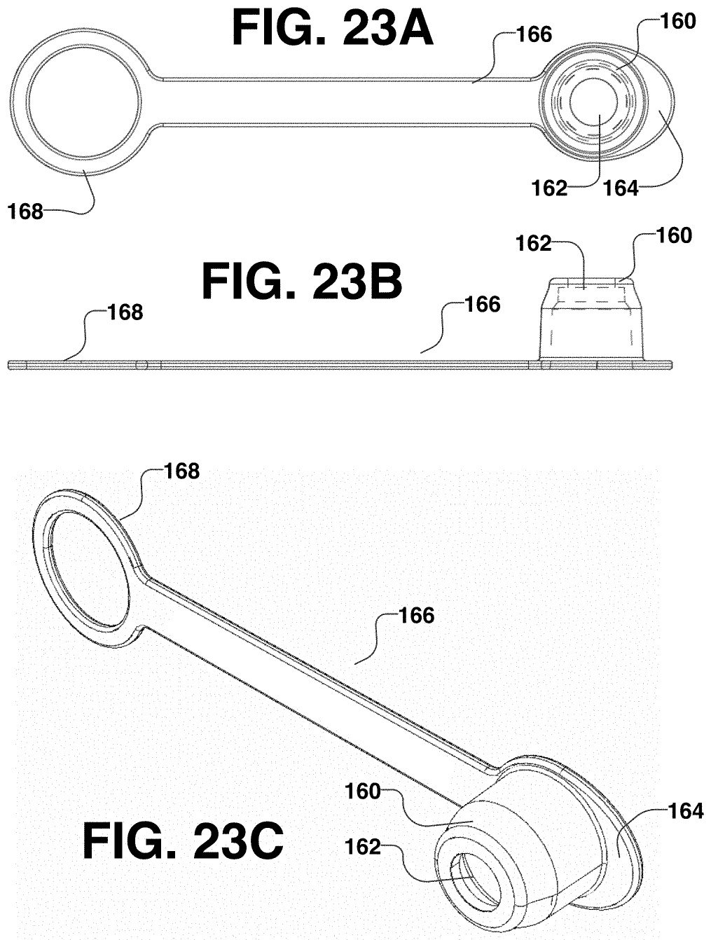

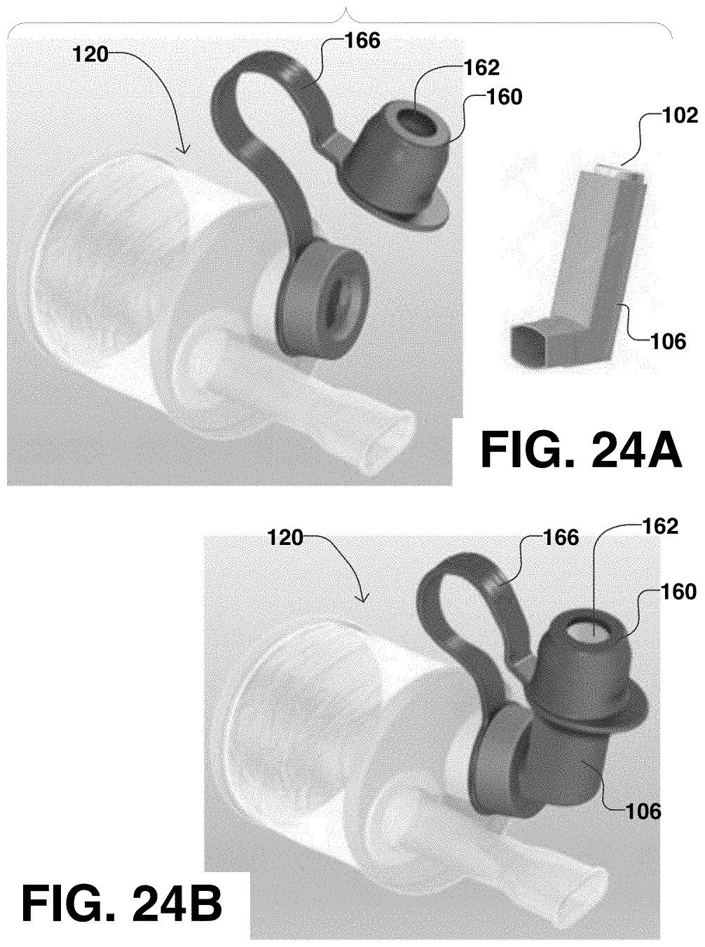

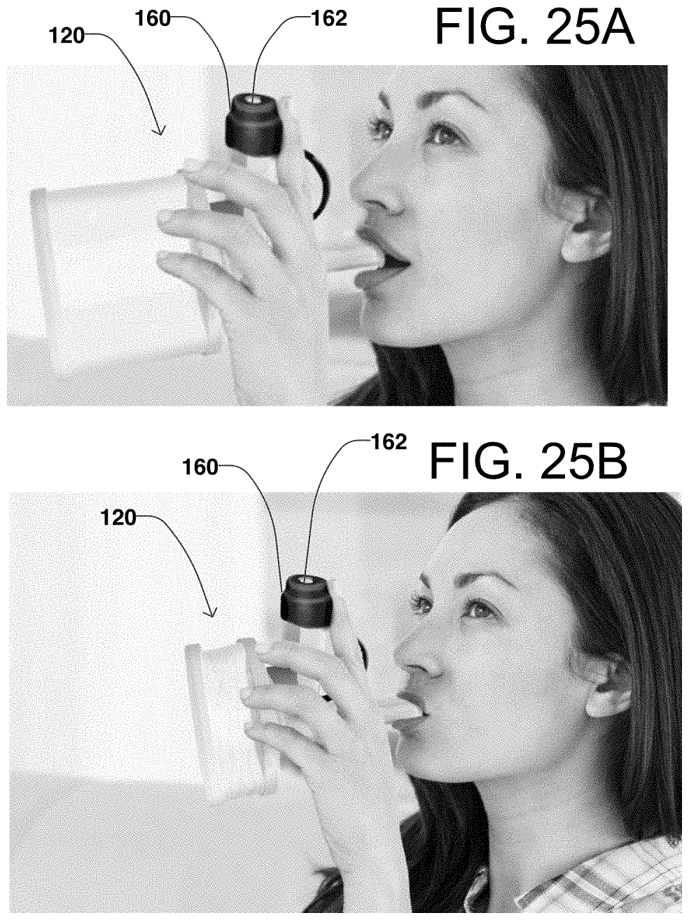

Since the cap 160 is generally to be used in combination with the device 120, the cap may be tethered to the device. For example, and with reference now to FIGS. 23A-23C, embodiments hereof may incorporate a flexible tether 166 having a cap 160 on one end thereof and a ring 168 on the other end. The ring 168 may be sized to fit between the collar 138 and the adaptor 142, with the ring being held in place by the adaptor 142 (as shown, e.g., in FIG. 24A). The cap 160 is sized to fit over the actuator/canister combination an MDI as described above (e.g., MDI 100′ as shown in FIG. 24B).

The tether 166 may be made with any suitable flexible material, preferably a material that is safe for skin contact. In a presently preferred exemplary implementation, the tether 166 is made from a viscoelastic PU self-skinning molded foam (at about 3 pounds per cubic foot) and is about 3 mm thick.

Using the Device

To use the device 120 with a particular MDI (e.g., MDI 100 shown in FIG. 1) the mouthpiece 122 is inserted via the locking tabs 150 into slots 132 and rotated to lock the mouthpiece to the top end cap 128. The actuator 106 is inserted into the innermost ring 158 so that it is properly aligned with the opening in the innermost ring 158 and the collar 138. The user fully opens the bag 146 and then depresses the canister 102, which then generates an aerosolized plume of medication into the bag 146. With the actuator exit tube 116 properly aligned in the collar 138, the plume 114 is directed into the bag 146, preferably toward the center of the bag 146.

The user then inhales through mouthpiece 122, generating negative pressure in the bag 146 and causing the aerosolized medication to flow via the mouthpiece 122 into the user's respiratory tract, thereby also collapsing the bag 146.

As the user inhales through mouthpiece 122, the vibrating members 154 of the flow reed attachment 124 signal if the user inhales above a predetermined flow rate (e.g., above 1.0 liter/sec).

After inhalation and a 10 second breath hold, the user manually opens and expands the bag 146 to allow for a subsequent MDI actuation cycle.

Usage with a Cap

To use the device 120 with a cap 160 and with a particular MDI (e.g., MDI 100 as shown in FIG. 1) the mouthpiece 122 is inserted via the locking tabs 150 into slots 132 and rotated to lock the mouthpiece to the top end cap 128. The actuator 106 is inserted into the innermost ring 158 so that it is properly aligned with the opening in the innermost ring 158 and the collar 138.

The cap 160 is positioned over the end of the actuator 106 and the associated canister 102, as shown in FIGS. 22B and 24B.

The user fully opens the bag 146 and then depresses the canister 102 through the hole 162 in the top of the cap 160, which then generates an aerosolized plume of medication into the bag 146. With the actuator exit tube 116 properly aligned in the collar 138, the plume 114 is directed into the bag 146, preferably toward the center of the bag 146.

The user then inhales through mouthpiece 122, generating negative pressure in the bag 146 and causing the aerosolized medication to flow via the mouthpiece 122 into the user's respiratory tract, thereby also collapsing the bag 146.

As the user inhales through mouthpiece 122, the vibrating members 154 of the flow reed attachment 124 signal if the user inhales above a predetermined flow rate (e.g., above 1.0 liter/sec).

After inhalation and a 10 second breath hold, the user manually opens and expands the bag 146 to allow for a subsequent MDI actuation cycle.

FIGS. 25A and 25B show the exemplary device of FIGS. 24A and 24B in use, where FIG. 25A shows the device in expanded mode (e.g., prior to the user's inhalation), and FIG. 25B shows the device closed (e.g., at or near the end of the user's inhalation).

The device 120 provides two indicators or signals of proper usage. The first signal is a visual signal that indicates whether the user has fully inhaled the medication. This is indicated by the user seeing that the bag is fully collapsed. The second indicator is an audio signal indicating if the user incorrectly inhaled the medication. This signal would be indicated by the reed in the device emitting a whistling or other audible sound if user inhales too fast for proper medication delivery to the lungs.

Thus is provided a delivery assist device for press-and-breathe metered dose inhalers for providing aerosolized drug to a user through inhalation that provides for the receipt of various sized original manufactured, pre-assembled MDIs.

The exemplary device provides two indicators for the user to ensure proper usage—that a full dose of medication is inhaled and that the rate of inhalation is not too fast (i.e., that the inhalation rate does not exceed a recommended flow rate).

### CONCLUSION

As used herein, including in the claims, the phrase “using” means “using at least,” and is not exclusive. Thus, e.g., the phrase “using X” means “using at least X.” Unless specifically stated by use of the word “only,” the phrase “using X” does not mean “using only X.”

In general, as used herein, including in the claims, unless the word “only” is specifically used in a phrase, it should not be read into that phrase.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

The present invention also covers the exact terms, features, values, and ranges, etc. in case these terms, features, values, and ranges etc. are used in conjunction with terms such as “about,” “around,” “generally,” “substantially,” “essentially,” “at least,” etc. (i.e., “about 3” shall also cover exactly 3 or “substantially constant” shall also cover exactly constant).

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

1. An accessory delivery device for a press-and-breathe metered dose inhaler (MDI), the MDI having a pressurized canister inserted in an actuator, the device comprising:
a flexible collapsible bag having an open top end and a closed bottom end;
a top end cap having a top surface, and having a first opening and a second opening in the top surface, the top end cap being connected to the open top end of the flexible collapsible bag;
a tubular mouthpiece having a proximal end suitable to place in a user's mouth, and a distal end mounted in the first opening of said top surface, the tubular mouthpiece in fluid communication with the flexible collapsible bag;
a warning indicator constructed and adapted to provide an audible signal based on a predetermined inhalation flow rate through the tubular mouthpiece is exceeded;
an upstanding collar in the second opening in the top end cap, the upstanding collar being disposed at a fixed angle offset from perpendicular to the top surface of the top end cap;
an adaptor mounted on the upstanding collar, the adaptor having a centrally disposed flexible member, an opening in the centrally disposed flexible member adapted to receive a first portion of the actuator of the press-and-breathe metered dose inhaler; and
a cap constructed and adapted to seal a gap between an inner wall of the actuator and the canister,
wherein, when activated, the press-and-breathe metered dose inhaler dispenses an aerosol spray containing a drug through the adaptor in a direction away from the top end cap and into the flexible collapsible bag.

2. The device of claim 1, wherein, when attached to the MDI, the cap prevents air flow through the gap between the inner wall of the actuator and the canister.

3. The device of claim 2, wherein, when attached to the MDI, the cap provides access to a top portion of the canister to allow user activation of the MDI.

4. The device of claim 1, further comprising a tether attached to the cap at a first end of the tether, wherein the tether comprises a ring at a seconds end thereof, said ring connecting said tether to said collar.

5. The device of claim 1, wherein tubular mouthpiece protrudes vertically from the top surface of the top end cap.

6. The device of claim 1, wherein the adaptor is constructed and adapted to fit multiple different sized inhaler actuators.

7. The device of claim 1, wherein the fixed angle is offset from perpendicular to the top surface of the top end cap when the device is in use.

8. The device of claim 1, wherein the first portion of the actuator comprises an actuator exit tube of the actuator.

9. The device of claim 1, and further comprising a flexible sealing member mounted in the centrally disposed flexible member having a slot to receive the press-and-breathe metered dose inhaler in fluid tight engagement.

10. The device of claim 1, and further comprising a bottom end cap sealing the bottom end of the flexible collapsible bag.

11. The device of claim 10, wherein the top end cap and bottom end cap comprise high density polyethylene.

12. The device of claim 1, wherein the warning indicator is mounted in the tubular mouthpiece.

13. The device of claim 1, wherein said warning indicator produces an audible sound when a flow rate of air passing through the tubular mouthpiece exceeds the predetermined inhalation flow rate.

14. The device of claim 13, wherein the warning indicator comprises a reed that vibrates to produce said audible sound when a flow rate of air passing through the tubular mouthpiece exceeds the predetermined inhalation flow rate.

15. The device of claim 13, wherein said warning indicator produces a whistling sound when the flow rate of air passing through the tubular mouthpiece from the flexible collapsible bag exceeds the predetermined inhalation flow rate.

16. The device of claim 1, wherein the flexible collapsible bag comprises low density polyethylene.

17. The device of claim 16, wherein the flexible collapsible bag comprises is metallocene low density polyethylene.

18. The device of claim 1, wherein the flexible collapsible bag provides a visual indication that an appropriate amount of the aerosol spray containing the drug has been inhaled when the flexible collapsible bag is collapsed.

19. The device of claim 1, and further comprising a channel in the press-and-breathe metered dose inhaler adaptor that receives the collar to securely attach the press-and-breathe metered dose inhaler adaptor to the collar.

20. (canceled)

21. The device of claim 1, wherein the fixed angle of the collar to the top surface of the top end cap is between 8 to 18 degrees.

22. The device of claim 1, wherein an airtight seal is provided between the top end cap and the open top end of the flexible collapsible bag.

23. The device of claim 22, wherein the airtight seal is provided by a seal surrounding the top end cap.

24. The device of claim 1, wherein the bottom end of the flexible collapsible bag is sealed to prevent passage of air into or out from the flexible collapsible bag from the bottom end.

25. The device of claim 1, further comprising:
a filter within the tubular mouthpiece.
